(19) United States
(12) Patent Application Publication
(10) Pub. No.: US 2017/0086871 A1
(43) Pub. Date: Mar. 30, 2017

(54) ASYMMETRIC MEMBRANE REMOVING FORCEPS

(71) Applicant: Katalyst Surgical, LLC, Chesterfield, MO (US)

(72) Inventors: Gregg D Scheller, Wildwood, MO (US); Eric J Bass, Webster Groves, MO (US)

(73) Assignee: Katalyst Surgical, LLC, Chesterfield, MO (US)

(21) Appl. No.: 15/253,916

(22) Filed: Sep. 1, 2016

Related U.S. Application Data
(60) Provisional application No. 62/222,828, filed on Sep. 24, 2015.

(51) Int. Cl. A61B 17/30

(52) U.S. Cl. CPC A61B 17/30; A61B 2017/305; A61B 2017/0046

(57) ABSTRACT

An asymmetric membrane removing forceps may include a stationary forceps jaw having a stationary forceps jaw distal end and a stationary forceps jaw proximal end and an actuating forceps jaw having an actuating forceps jaw distal end and an actuating forceps jaw proximal end. A compression of an actuation structure may be configured to extend a hypodermic tube over a portion of the stationary forceps jaw and over a portion of the actuating forceps jaw. An extension of the hypodermic tube over the portion of the stationary forceps jaw and over the portion of the actuating forceps jaw may be configured to actuate the actuating forceps jaw relative to the stationary forceps jaw. The extension of the hypodermic tube over the portion of the stationary forceps jaw and over the portion of the actuating forceps jaw may be configured not to actuate the stationary forceps jaw.

### CROSS-REFERENCE TO RELATED APPLICATIONS

This Application claims the benefit of U.S. Provisional Application No. 62/222,828, filed Sep. 24, 2015.

### FIELD OF THE INVENTION

The present disclosure relates to a surgical instrument, and, more particularly, to a microsurgical forceps.

### BACKGROUND OF THE INVENTION

A microsurgical forceps may be used to perform a microsurgical procedure, e.g., an ophthalmic surgical procedure. For example, a surgeon may use a forceps to grasp and manipulate tissues or other surgical instruments to perform portions of a surgical procedure. A particular microsurgical procedure may require a surgeon to separate a first tissue from a second tissue without causing trauma to at least one of the tissues. Such a separation procedure may be particularly difficult for a surgeon to perform if the tissue surface geometry is not flat, e.g., if the tissue surface geometry is convex. For example, an ophthalmic surgeon may be required to remove an internal limiting membrane from a patient's retina without causing trauma to the patient's retina. Accordingly, there is a need for a microsurgical forceps that enables a surgeon to separate a first tissue from a second tissue without causing significant trauma to at least one of the tissues.

### BRIEF SUMMARY OF THE INVENTION

The present disclosure provides an asymmetric membrane removing forceps. In one or more embodiments, an asymmetric membrane removing forceps may comprise a stationary forceps jaw having a stationary forceps jaw distal end and a stationary forceps jaw proximal end and an actuating forceps jaw having an actuating forceps jaw distal end and an actuating forceps jaw proximal end. Illustratively, a compression of an actuation structure may be configured to extend a hypodermic tube over a portion of the stationary forceps jaw and over a portion of the actuating forceps jaw. In one or more embodiments, an extension of the hypodermic tube over the portion of the stationary forceps jaw and over the portion of the actuating forceps jaw may be configured to actuate the actuating forceps jaw relative to the stationary forceps jaw. Illustratively, the extension of the hypodermic tube over the portion of the stationary forceps jaw and over the portion of the actuating forceps jaw may be configured not to actuate the stationary forceps jaw.

### BRIEF DESCRIPTION OF THE DRAWINGS

The above and further advantages of the present invention may be better understood by referring to the following description in conjunction with the accompanying drawings in which like reference numerals indicate identical or functionally similar elements:

FIGS. 1A, 1B, 1C, 1D, 1E, 1F, and 1G are schematic diagrams illustrating an asymmetric membrane removing forceps tip;

FIG. 2A is a schematic diagram illustrating an exploded view of an asymmetric membrane removing forceps assembly;

FIGS. 2B and 2C are schematic diagrams illustrating an assembled asymmetric membrane removing forceps;

FIGS. 3A, 3B, and 3C are schematic diagrams illustrating a gradual closing of an asymmetric membrane removing forceps tip;

FIGS. 4A, 4B, and 4C are schematic diagrams illustrating a gradual closing of an asymmetric membrane removing forceps tip;

FIGS. 5A, 5B, and 5C are schematic diagrams illustrating a surgical procedure.

### DETAILED DESCRIPTION OF AN ILLUSTRATIVE EMBODIMENT

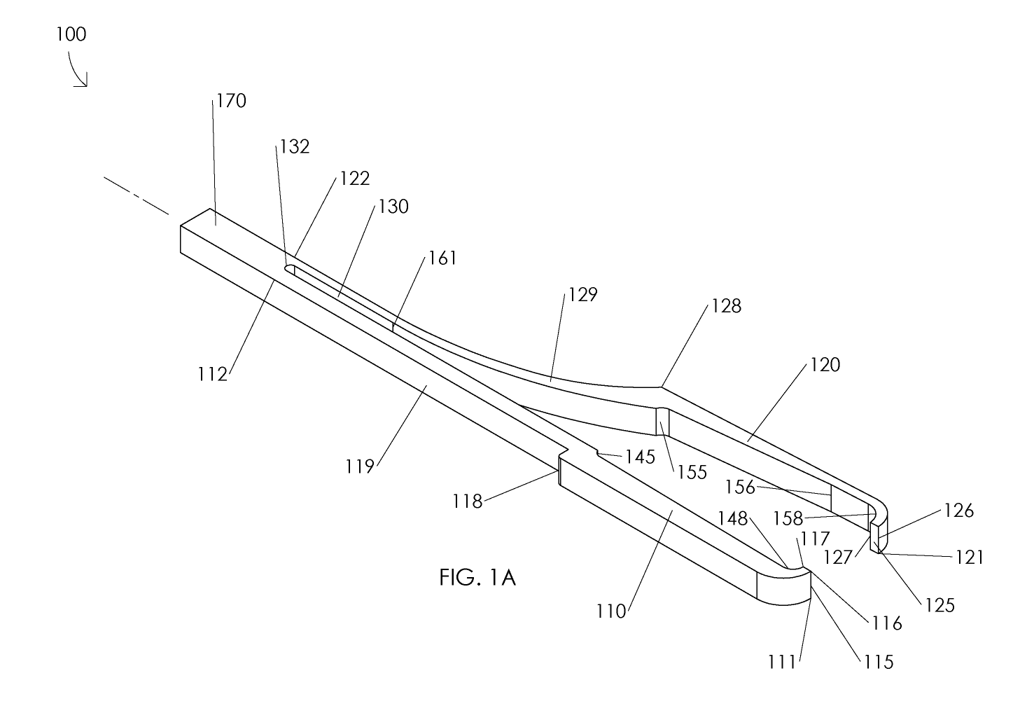

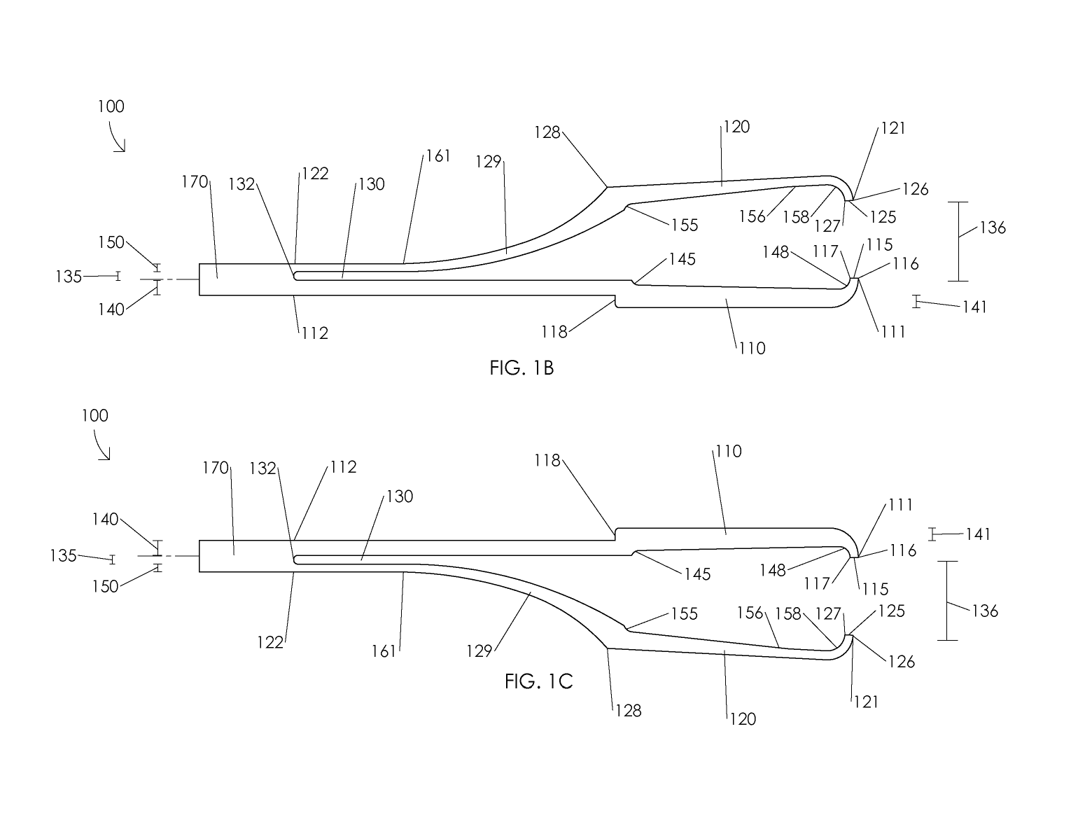

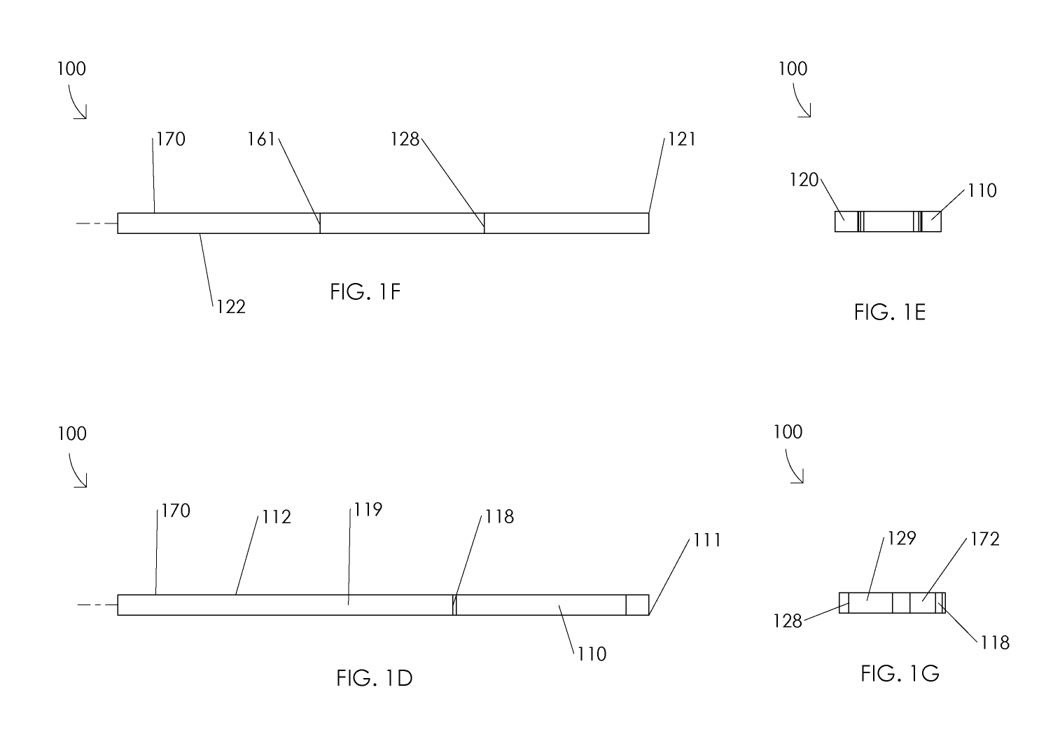

FIGS. 1A, 1B, 1C, 1D, 1E, 1F, and 1G are schematic diagrams illustrating an asymmetric membrane removing forceps tip 100. FIG. 1A illustrates an isometric view of an asymmetric membrane removing forceps tip 100. Illustratively, asymmetric membrane removing forceps tip 100 may comprise a stationary forceps jaw 110 having a stationary forceps jaw distal end 111 and a stationary forceps jaw proximal end 112 and an actuating forceps jaw 120 having an actuating forceps jaw distal end 121 and an actuating forceps jaw proximal end 122. In one or more embodiments, stationary forceps jaw 110 may comprise a stationary medial face 115, a stationary forceps jaw distal shoulder 148, a stationary forceps jaw proximal shoulder 145, a lateral projection 118, and a stationary base 119. Illustratively, stationary medial face 115 may comprise a stationary medial face distal end 116 and a stationary medial face proximal end 117. In one or more embodiments, stationary medial face distal end 116 and stationary medial face proximal end 117 may be separated by a distance in a range of 0.002 to 0.004 inches, e.g., stationary medial face distal end 116 and stationary medial face proximal end 117 may be separated by a distance of 0.0025 inches. Illustratively, stationary medial face distal end 116 and stationary medial face proximal end 117 may be separated by a distance of less than 0.002 inches or greater than 0.004 inches.

In one or more embodiments, actuating forceps jaw 120 may comprise an actuating medial face 125, an actuating forceps jaw distal shoulder 158, an actuating forceps jaw proximal shoulder 155, an actuating forceps jaw medial discontinuity 156, and an actuating curved base 129. Illustratively, actuating medial face 125 may comprise an actuating medial face distal end 126 and an actuating medial face proximal end 127. In one or more embodiments, actuating medial face distal end 126 and an actuating medial face proximal end 127 may be separated by a distance in a range of 0.002 to 0.004 inches, e.g., actuating medial face distal end 126 and an actuating medial face proximal end 127 may be separated by a distance of 0.0025 inches. Illustratively, actuating medial face distal end 126 and an actuating medial face proximal end 127 may be separated by a distance of less than 0.002 inches or greater than 0.004 inches. In one or more embodiments, actuating curved base 129 may comprise an actuating curved base distal end 128 and an actuating curved base proximal end 161. Illustratively, asymmetric membrane removing forceps tip 100 may comprise an asymmetric aperture 130 having an asymmetric aperture proximal end 132. In one or more embodiments, asymmetric aperture 130 may be disposed between a portion of stationary forceps jaw 110 and a portion of actuating forceps jaw 120.

FIG. 1B illustrates a top view of an asymmetric membrane removing forceps tip 100. FIG. 1C illustrates a bottom view of an asymmetric membrane removing forceps tip 100. Illustratively, asymmetric membrane removing forceps tip 100 may comprise an asymmetric aperture width 135. In one or more embodiments, asymmetric aperture width 135 may comprise a distance in a range of 0.001 to 0.004 inches, e.g., asymmetric aperture width 135 may comprise a distance of 0.003 inches. Illustratively, asymmetric aperture width 135 may comprise a distance of less than 0.001 inches or greater than 0.004 inches. In one or more embodiments, asymmetric membrane removing forceps tip 100 may comprise a medial face separation distance 136. Illustratively, medial face separation distance 136 may be configured to facilitate an insertion of asymmetric membrane removing forceps tip 100 into an ophthalmic cannula, e.g., a valved cannula. In one or more embodiments, medial face separation distance 136 may comprise a first distance and an inner diameter of an ophthalmic cannula may comprise a second distance. Illustratively, the first distance may be less than the second distance. In one or more embodiments, medial face separation distance 136 may comprise a distance in a range of 0.021 to 0.033 inches, e.g., medial face separation distance 136 may comprise a distance of 0.025 inches. Illustratively, medial face separation distance 136 may comprise a distance of less than 0.021 inches or greater than 0.033 inches. In one or more embodiments, asymmetric membrane removing forceps tip 100 may comprise a stationary forceps jaw proximal width 140. Illustratively, stationary forceps jaw proximal width 140 may comprise a distance in a range of 0.0046 to 0.0058 inches, e.g., stationary forceps jaw proximal width 140 may comprise a distance of 0.0051 inches. In one or more embodiments, stationary forceps jaw proximal width 140 may comprise a distance of less than 0.0046 inches or greater than 0.0058 inches. Illustratively, asymmetric membrane removing forceps tip 100 may comprise a lateral projection distance 141. In one or more embodiments, lateral projection distance 141 may comprise a distance in a range of 0.0041 to 0.0054 inches, e.g., lateral projection distance 141 may comprise a distance of 0.0045 inches. Illustratively, lateral projection distance 141 may comprise a distance of less than 0.0041 inches or greater than 0.0054 inches. In one or more embodiments, asymmetric membrane removing forceps tip 100 may comprise an actuating forceps jaw proximal width 150. Illustratively, actuating forceps jaw proximal width 150 may comprise a distance in a range of 0.002 to 0.0035 inches, e.g., actuating forceps jaw proximal width 150 may comprise a distance of 0.0025 inches. In one or more embodiments, actuating forceps jaw proximal width 150 may comprise a distance of less than 0.002 inches or greater than 0.0035 inches.

Illustratively, asymmetric aperture 130 may be cut off-axis in blank 170, e.g., asymmetric aperture 130 may be cut wherein asymmetric aperture 130 may be disposed laterally relative to a midline of blank 170. In one or more embodiments, disposing asymmetric aperture 130 off-axis relative to a midline of blank 170 may be configured to increase a grasping force between actuating forceps jaw 120 and stationary forceps jaw 110. Illustratively, disposing asymmetric aperture 130 off-axis relative to a midline of blank 170 may be configured to increase a grasping force between actuating medial face 125 and stationary medial face 115. In one or more embodiments, asymmetric aperture proximal end 132 may be disposed between stationary forceps jaw proximal end 112 and actuating forceps jaw proximal end 122. Illustratively, actuating curved base proximal end 161 may be disposed between actuating forceps jaw proximal end 122 and actuating curved base distal end 128. In one or more embodiments, actuating forceps jaw medial discontinuity 156 may be disposed between actuating forceps jaw distal shoulder 158 and actuating forceps jaw proximal shoulder 155, e.g., actuating forceps jaw medial discontinuity 156 may be disposed between actuating forceps jaw proximal shoulder 155 and actuating forceps jaw distal end 121. Illustratively, actuating curved base distal end 128 may be disposed between actuating forceps jaw medial discontinuity 156 and actuating curved base proximal end 161. In one or more embodiments, lateral projection 118 may be disposed between stationary forceps jaw distal shoulder 148 and stationary forceps jaw proximal end 112. Illustratively, lateral projection 118 may extend out from stationary base 119 at an angle normal to a surface of stationary base 119, e.g., lateral projection 118 may extend out from stationary base 119 at a 90.0 degree angle relative to a surface of stationary base 119. In one or more embodiments, stationary forceps jaw proximal shoulder 145 may be disposed between stationary forceps jaw proximal end 122 and stationary forceps jaw distal shoulder 148. Illustratively, stationary forceps jaw distal shoulder 148 may be disposed between stationary forceps jaw proximal shoulder 145 and stationary forceps jaw distal end 111.

FIG. 1D illustrates a side view of an asymmetric membrane removing forceps tip 100. FIG. 1E illustrates a front view of an asymmetric membrane removing forceps tip 100. FIG. 1F illustrates a side view of an asymmetric membrane removing forceps tip 100 rotated 180 degrees. FIG. 1G illustrates a back view of an asymmetric membrane removing forceps tip 100. In one or more embodiments, asymmetric membrane removing forceps tip 100 may be manufactured from any suitable material, e.g., polymers, metals, metal alloys, etc., or from any combination of suitable materials. Illustratively, asymmetric membrane removing forceps tip 100 may be manufactured with dimensions configured for performing microsurgical procedures, e.g., ophthalmic surgical procedures. In one or more embodiments, asymmetric membrane removing forceps tip 100 may be manufactured from a blank 170. In one or more embodiments, asymmetric membrane removing forceps tip 100 may be manufactured by modifying blank 170, e.g., with an electric discharge machine, a laser, a file, deep reactive ion etching, or any suitable modification means. Illustratively, asymmetric membrane removing forceps tip 100 may be manufactured by a 3D printing process. For example, asymmetric membrane removing forceps tip 100 may be manufactured by selective laser sintering, selective heat sintering, selective laser melting, electron-beam melting, direct metal laser sintering, electron beam freeform fabrication, etc.

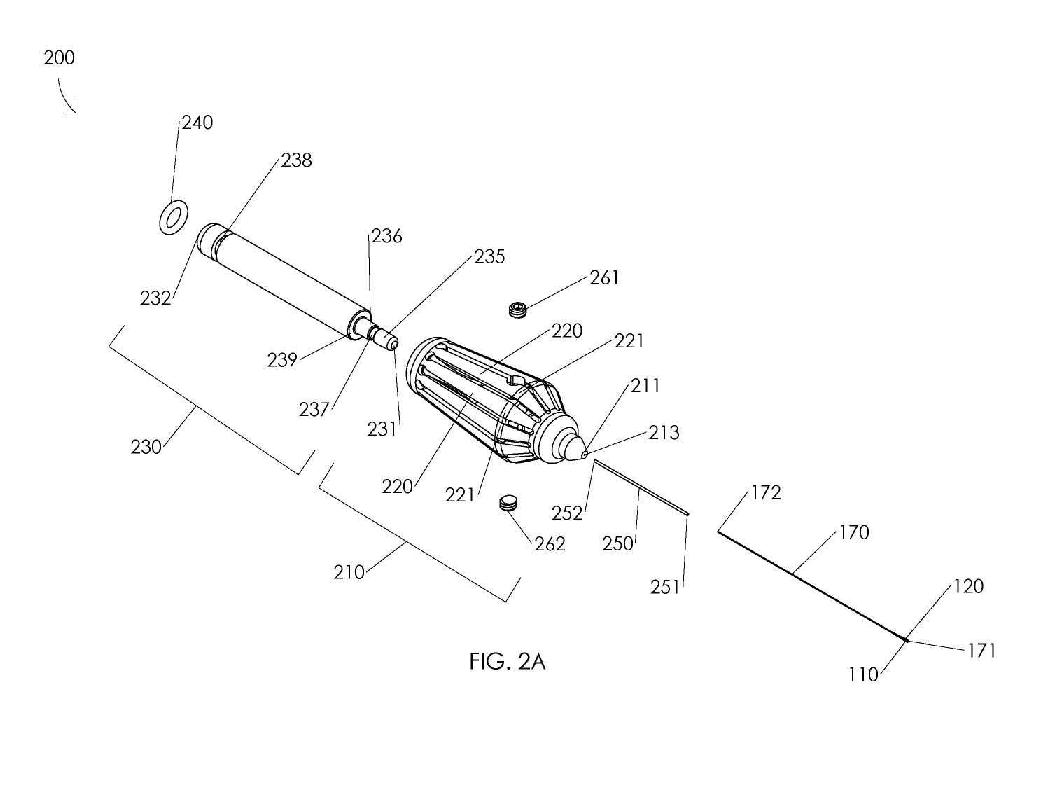

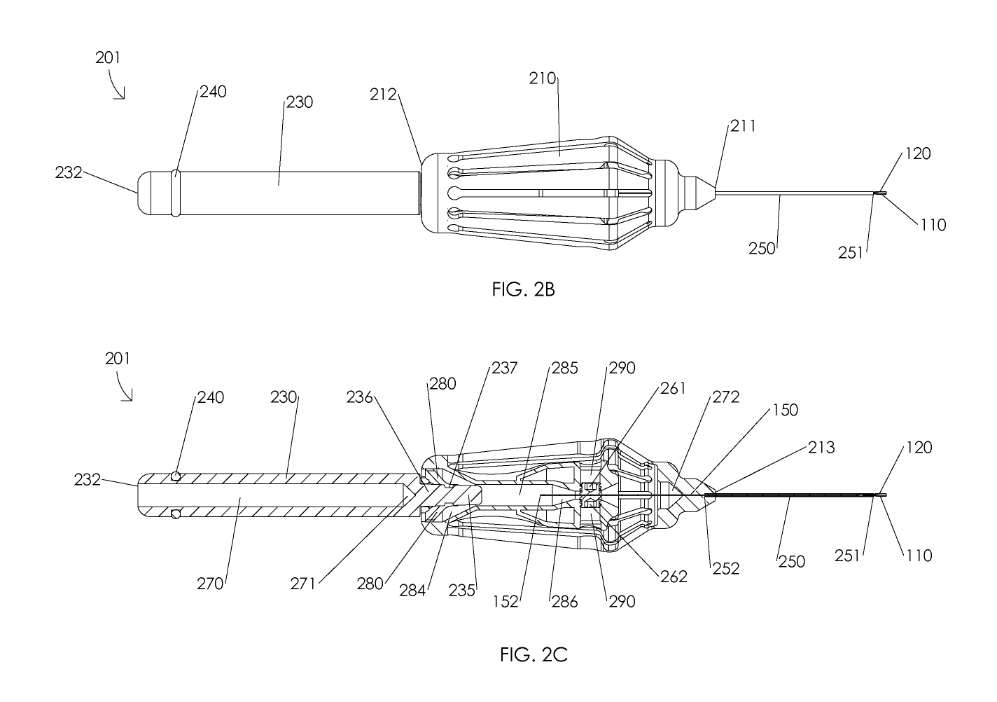

FIG. 2A is a schematic diagram illustrating an exploded view of an asymmetric membrane removing forceps assembly 200. Illustratively, an asymmetric membrane removing forceps assembly 200 may comprise an actuation structure 210, a removable handle 230, a gage indicator 240, a hypodermic tube 250, a blank 170, a superior setscrew 261, and an inferior setscrew 262. In one or more embodiments, an actuation structure 210 may comprise an actuation structure distal end 211, an actuation structure proximal end 212, a plurality of actuation arms 220, and a hypodermic tube housing 213. Illustratively, each actuation arm 220 of actuation structure 210 may comprise at least one extension mechanism 221. In one or more embodiments, removable handle 230 may comprise a removable handle distal end 231, a removable handle proximal end 232, a barb head 235, a barb base 236, a barb channel 237, a gage indicator housing 238, and an actuation structure interface 239. Illustratively, hypodermic tube 250 may comprise a hypodermic tube distal end 251 and a hypodermic tube proximal end 252. In one or more embodiments, blank 170 may comprise a blank distal end 171, a blank proximal end 172, and an asymmetric membrane removing forceps tip 100.

FIGS. 2B and 2C are schematic diagrams illustrating an assembled asymmetric membrane removing forceps 201. FIG. 2B illustrates a side view of an assembled asymmetric membrane removing forceps 201. FIG. 2C illustrates a cross-sectional view of an assembled asymmetric membrane removing forceps 201. Illustratively, gage indicator 240 may be disposed within gage indicator housing 238. In one or more embodiments, gage indicator 240 may be configured to visually indicate a size of hypodermic tube 250, e.g., gage indicator 240 may comprise a ring colored to visually indicate an outer diameter of hypodermic tube 250. Illustratively, removable handle 230 may comprise an inner bore 270 and an inner bore distal taper 271. In one or more embodiments, inner bore 270 may have an inner bore distal end and an inner bore proximal end wherein the inner bore proximal end is adjacent to the removable handle proximal end 232 Illustratively, inner bore distal taper 271 may be disposed between a distal end of inner bore 270 and barb base 236. In one or more embodiments, actuation structure 210 may comprise an inner nosecone 272, a plurality of fingers 280, an inner chamber proximal taper 284, an inner chamber 285, an inner chamber distal taper 286, and a setscrew housing 290. Illustratively, inner nosecone 272 may be disposed between setscrew housing 290 and actuation structure distal end 211. In one or more embodiments, setscrew housing 290 may be disposed between inner chamber distal taper 286 and inner nosecone 272. Illustratively, inner chamber distal taper 286 may be disposed between inner chamber 285 and setscrew housing 290. In one or more embodiments, inner chamber 285 may be disposed between inner chamber proximal taper 284 and inner chamber distal taper 286. Illustratively, each finger 280 of the plurality of fingers 280 may be disposed in inner chamber proximal taper 284.

Illustratively, a portion of removable handle 230 may be disposed within a portion of actuation structure 210, e.g., removable handle distal end 231 may be disposed within actuation structure 210. In one or more embodiments, barb head 235 may be disposed within actuation structure 210 wherein barb head 235 is disposed in inner chamber 285 and inner chamber proximal taper 284. Illustratively, barb base 236 may be disposed within actuation structure 210 wherein barb base 236 is disposed in inner chamber proximal taper 284. In one or more embodiments, barb channel 237 may be disposed within actuation structure 210 wherein barb channel 237 is disposed in inner chamber proximal taper 284. Illustratively, each finger 280 of the plurality of fingers 280 may be partially disposed in barb channel 237.

In one or more embodiments, a portion of removable handle 230 may be temporarily fixed within actuation structure 210, e.g., barb head 235, barb base 236, and barb channel 237 may be temporarily fixed within actuation structure 210. Illustratively, each finger 280 of the plurality of fingers 280 may be configured to temporarily fix a portion of removable handle 230 within actuation structure 210. In one or more embodiments, each finger 280 of the plurality of fingers 280 may be configured to temporarily fix a portion of removable handle 230 within actuation structure 210 by a snap fit, e.g., each finger 280 of the plurality of fingers 280 may be configured to temporarily fix a portion of removable handle 230 within actuation structure 210 by a torsional snap fit. Illustratively, a portion of removable handle 230 may be temporarily fixed within actuation structure 210 by a force of friction, e.g., a portion of removable handle 230 may be temporarily fixed within actuation structure 210 by an interference fit. In one or more embodiments, a portion of removable handle 230 may be disposed within a portion of actuation structure 210 wherein actuation structure interface 239 is adjacent to actuation structure proximal end 212.

Illustratively, a surgeon may optionally remove a portion of removable handle 230 from a portion of actuation structure 210. For example, a surgeon may optionally remove removable handle 230 from actuation structure 210 to grasp actuation structure wherein a portion of the surgeon's palm is adjacent to actuation structure proximal end 212. In one or more embodiments, a surgeon may optionally remove removable handle 230 from actuation structure 210 by pulling removable handle 230 out from inner chamber proximal taper 284. Illustratively, a surgeon may optionally insert removable handle 230 into actuation structure 210 by pushing removable handle 230 into inner chamber proximal taper 284. In one or more embodiments, a surgeon may perform a first portion of a surgical procedure with removable handle 230 disposed within actuation structure 210. Illustratively, the surgeon may perform a second portion of the surgical procedure with removable handle 230 removed from actuation structure 210. In one or more embodiments, the surgeon may perform a third portion of the surgical procedure with removable handle 230 disposed within actuation structure 210. Illustratively, the surgeon may perform a fourth portion of the surgical procedure with removable handle 230 removed from actuation structure 210.

In one or more embodiments, a portion of hypodermic tube 250 may be disposed in a portion of actuation structure 210, e.g., hypodermic tube proximal end 251 may be disposed in a portion of actuation structure 210. Illustratively, a portion of hypodermic tube 250 may be disposed in hypodermic tube housing 213, e.g., hypodermic tube proximal end 252 may be disposed in hypodermic tube housing 213. In one or more embodiments, a portion of hypodermic tube 250 may be fixed within a portion of actuation structure 210, e.g., a portion of hypodermic tube 250 may be fixed within a portion of actuation structure 210 by an adhesive, a weld, a force of friction, etc.

Illustratively, blank 170 may be disposed in hypodermic tube 250 and actuation structure 210, e.g., blank 170 may be disposed in hypodermic tube 250 an actuation structure 210 wherein blank proximal end 172 is disposed in actuation structure 210 and blank distal end 171 extends from hypodermic tube distal end 251. In one or more embodiments, blank 170 may be disposed in hypodermic tube 250, inner nosecone 272, setscrew housing 290, inner chamber distal taper 286, and inner chamber 285. Illustratively, superior setscrew 261 and inferior setscrew 262 may be disposed within setscrew housing 290. In one or more embodiments, blank 170 may be fixed in a position relative to actuation structure proximal end 212 and hypodermic tube 250, e.g., superior setscrew 261 and inferior setscrew 262 may be configured to fix blank 170 in a position relative to actuation structure proximal end 212 and hypodermic tube 250. Illustratively, a portion of blank 170 may be disposed between superior setscrew 261 and inferior setscrew 262 wherein the portion of blank 170 is fixed in a position relative to actuation structure proximal end 212 and hypodermic tube 250 by a force applied to the portion of blank 170 by superior setscrew 261 and inferior setscrew 262.

In one or more embodiments, a compression of actuation structure 210 may be configured to extend actuation structure distal end 211 relative to actuation structure proximal end 212. Illustratively, a compression of actuation structure 210 may be configured to extend hypodermic tube 250 relative to blank 170. In one or more embodiments, a compression of actuation structure 210 may be configured to extend hypodermic tube distal end 251 over a portion of stationary forceps jaw 110 and over a portion of actuating forceps jaw 120, e.g., a compression of actuation structure 210 may be configured to extend hypodermic tube distal end 251 over a portion of actuating curved base 129. Illustratively, a compression of actuation structure 210 may be configured to decrease a distance between stationary medial face 115 and actuating medial face 125. For example, a compression of actuation structure 210 may be configured to actuate actuating medial face 125 relative to stationary medial face 115. In one or more embodiments, a compression of actuation structure 210 may be configured to close asymmetric membrane removing forceps tip 100.

In one or more embodiments, a decompression of actuation structure 210 may be configured to retract actuation structure distal end 211 relative to actuation structure proximal end 212. Illustratively, a decompression of actuation structure 210 may be configured to retract hypodermic tube 250 relative to blank 170. In one or more embodiments, a decompression of actuation structure 210 may be configured to retract hypodermic tube distal end 251 off from a portion of stationary forceps jaw 110 and off from a portion of actuating forceps jaw 120, e.g., a decompression of actuation structure 210 may be configured to retract hypodermic tube distal end 251 off from a portion of actuating curved base 129. Illustratively, a decompression of actuation structure 210 may be configured to increase a distance between stationary medial face 115 and actuating medial face 125. For example, a decompression of actuation structure 210 may be configured to actuate actuating medial face 125 relative to stationary medial face 115. In one or more embodiments, a decompression of actuation structure 210 may be configured to open asymmetric membrane removing forceps tip 100.

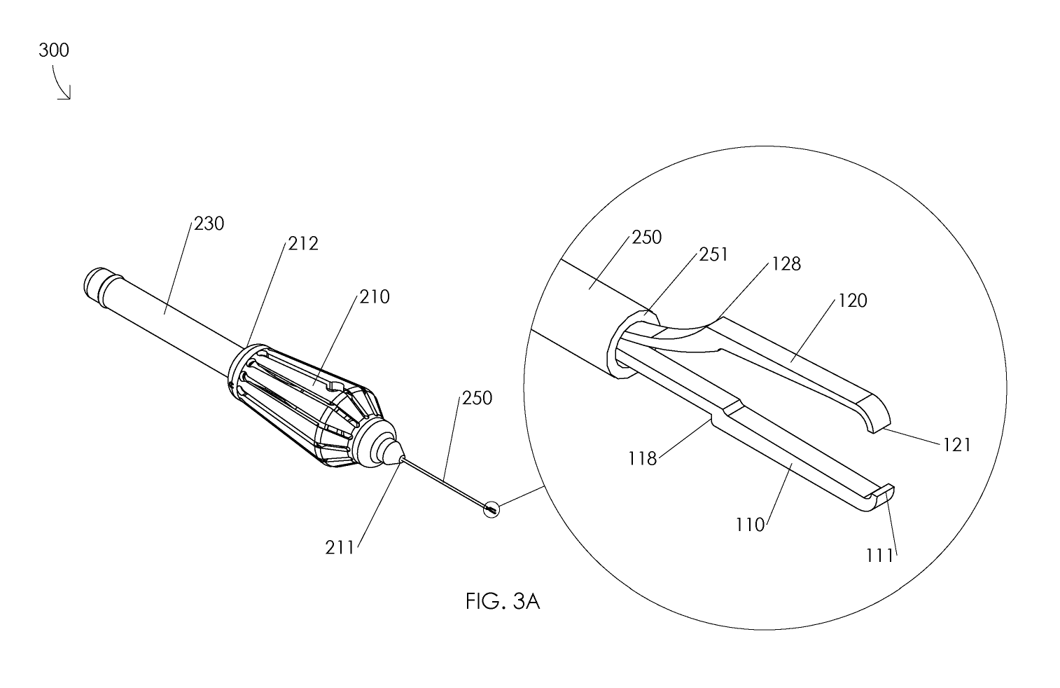

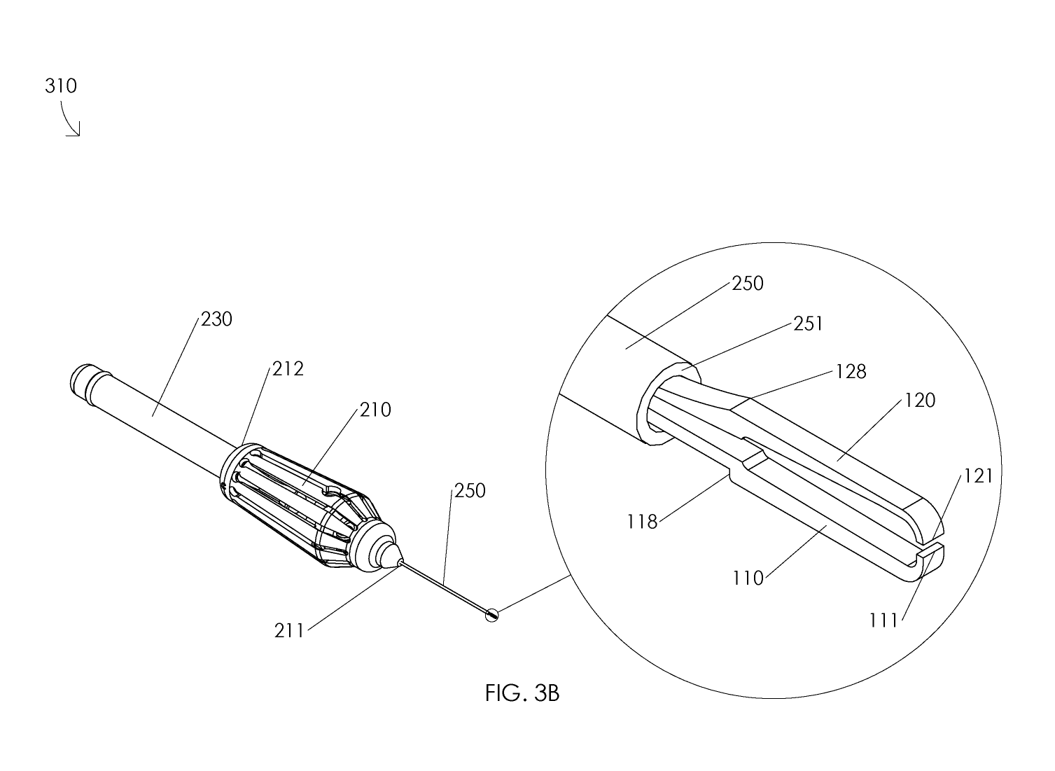

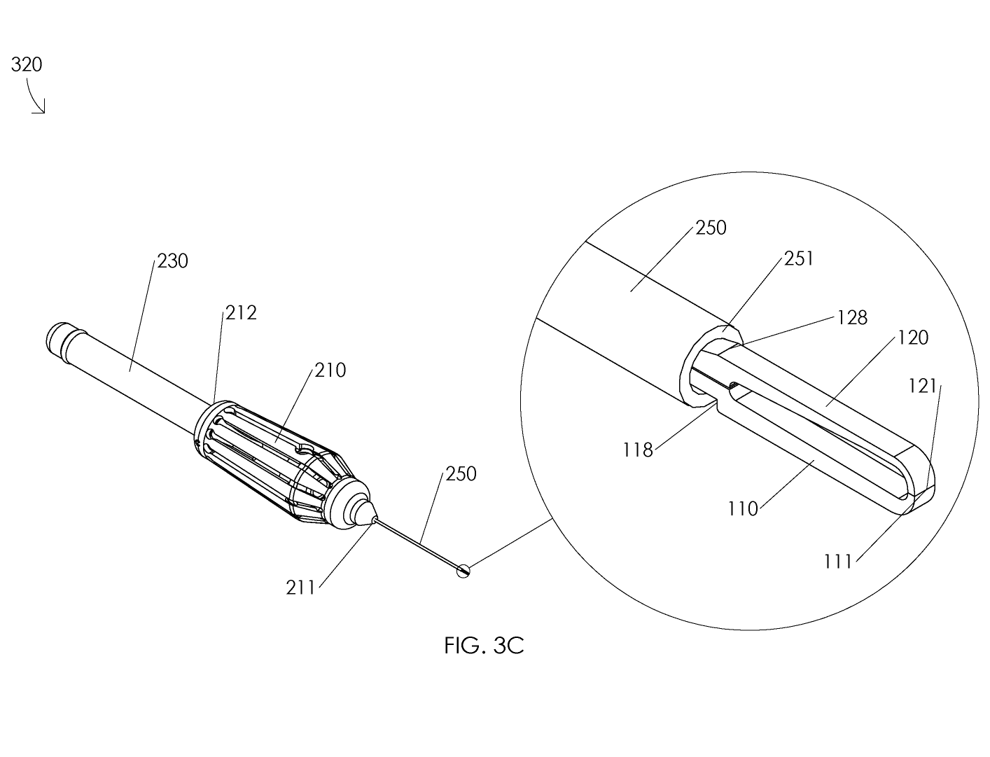

FIGS. 3A, 3B, and 3C are schematic diagrams illustrating a gradual closing of an asymmetric membrane removing forceps tip 100. FIG. 3A illustrates an isometric view of an open asymmetric membrane removing forceps 300. Illustratively, asymmetric membrane removing forceps tip 100 may comprise an open asymmetric membrane removing forceps 300 when actuation structure 210 is fully decompressed. In one or more embodiments, asymmetric membrane removing forceps tip 100 may comprise an open asymmetric membrane removing forceps 300 when stationary medial face 115 is fully separated from actuating medial face 125, e.g., asymmetric membrane removing forceps tip 100 may comprise an open asymmetric membrane removing forceps 300 when stationary medial face 115 is separated from actuating medial face 125 by medial face separation distance 136. Illustratively, a line normal to a surface of actuating medial face 125 may intersect a line normal to a surface of stationary medial face 115 at an angle when asymmetric membrane removing forceps tip 100 comprises an open asymmetric membrane removing forceps 300.

is FIG. 3B illustrates an isometric view of a partially closed asymmetric membrane removing forceps 310. Illustratively, a compression of actuation structure 210 may be configured to gradually close asymmetric membrane removing forceps tip 100 from an open asymmetric membrane removing forceps 300 to a partially closed asymmetric membrane removing forceps 310, e.g., asymmetric membrane removing forceps tip 100 may comprise a partially closed asymmetric removing forceps 310 when actuation structure 210 is partially compressed. In one or more embodiments, a compression of actuation structure 210 may be configured to extend hypodermic tube 250 over a portion of stationary forceps jaw 110 and over a portion of actuating forceps jaw 120. Illustratively, an extension of hypodermic tube 250 over a portion of stationary forceps jaw 110 and over a portion of actuating forceps jaw 120 may be configured to actuate actuating forceps jaw 120 relative to stationary forceps jaw 110. In one or more embodiments, a compression of actuation structure 210 may be configured to actuate actuating forceps jaw 120 relative to stationary forceps jaw 110 wherein a separation distance between stationary medial face 115 and actuating medial face 125 is gradually decreased. Illustratively, a compression of actuation structure 210 may be configured to gradually close asymmetric membrane removing forceps tip 100 wherein only actuating forceps jaw 120 actuates in a transverse plane of actuation structure 210, e.g., a compression of actuation structure 210 may be configured to gradually close asymmetric membrane removing forceps tip 100 wherein stationary forceps jaw 110 does not actuate in a transverse plane of actuation structure 210. In one or more embodiments, a line normal to a surface of actuating medial face 125 may be parallel to a line normal to a surface of stationary medial face 115 when asymmetric membrane removing forceps tip 100 comprises a partially closed asymmetric removing forceps 310. Illustratively, a compression of actuation structure 210 may be configured to cause a line normal to a surface of actuating medial face 125 that intersects a line normal to a surface of stationary medial face 115 at an angle to gradually become parallel to the line normal to the surface of stationary medial face 115.

FIG. 3C illustrates an isometric view of a fully closed asymmetric membrane removing forceps 320. Illustratively, a compression of actuation structure 210 may be configured to gradually close asymmetric membrane removing forceps tip 100 from a partially closed asymmetric membrane removing forceps 310 to a fully closed asymmetric membrane removing forceps 320, e.g., asymmetric membrane removing forceps tip 100 may comprise a fully closed asymmetric removing forceps 320 when actuation structure 210 is fully compressed. In one or more embodiments, actuating medial face 125 may contact stationary medial face 115 when asymmetric membrane removing forceps tip 100 comprises a fully closed asymmetric removing forceps 320. Illustratively, actuating medial face 125 may contact stationary medial face 115 wherein actuating medial face distal end 126 is adjacent to stationary medial face distal end 116 when asymmetric membrane removing forceps tip 100 comprises a fully closed asymmetric removing forceps 320. In one or more embodiments, actuating medial face 125 may contact stationary medial face 115 wherein actuating medial face proximal end 127 is adjacent to stationary medial face proximal end 117 when asymmetric membrane removing forceps tip 100 comprises a fully closed asymmetric removing forceps 320. Illustratively, actuating medial face 125 may contact stationary medial face 115 wherein actuating medial face distal end 126 is adjacent to stationary medial face distal end 116 and wherein actuating medial face proximal end 127 is adjacent to stationary medial face proximal end 117 when asymmetric membrane removing forceps tip 100 comprises a fully closed asymmetric removing forceps 320.

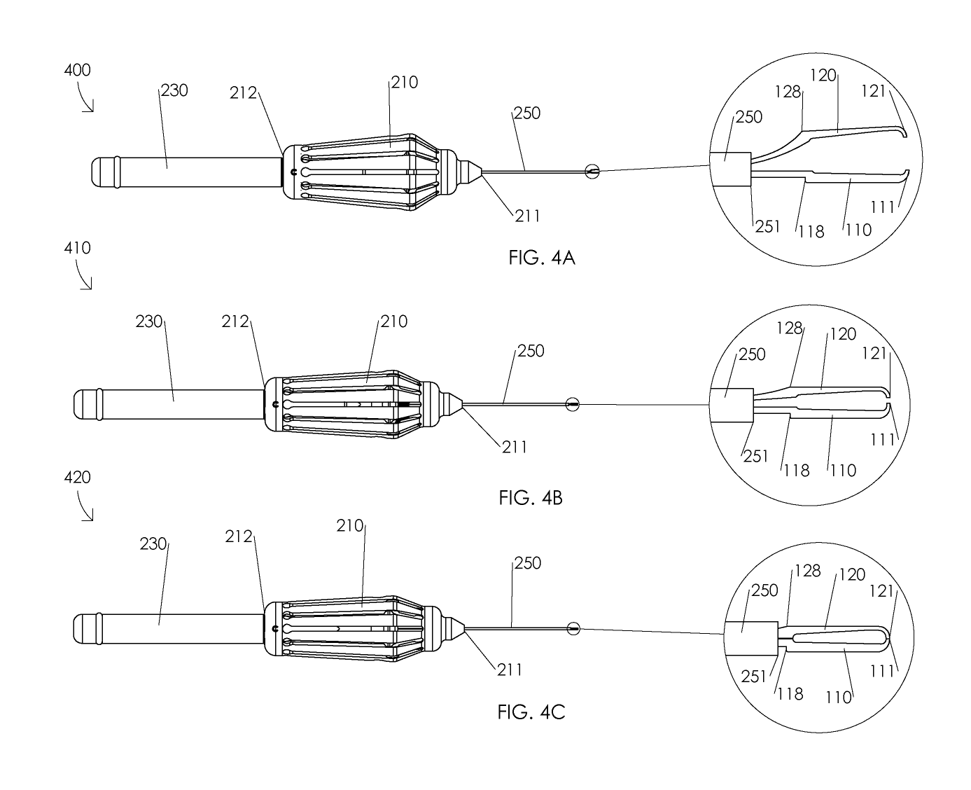

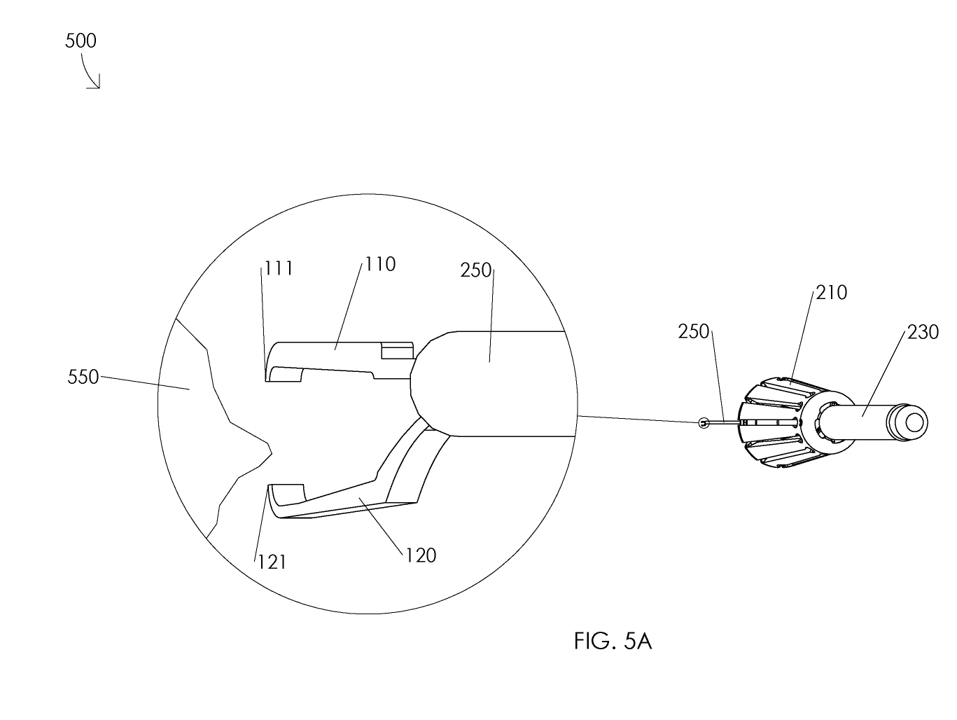

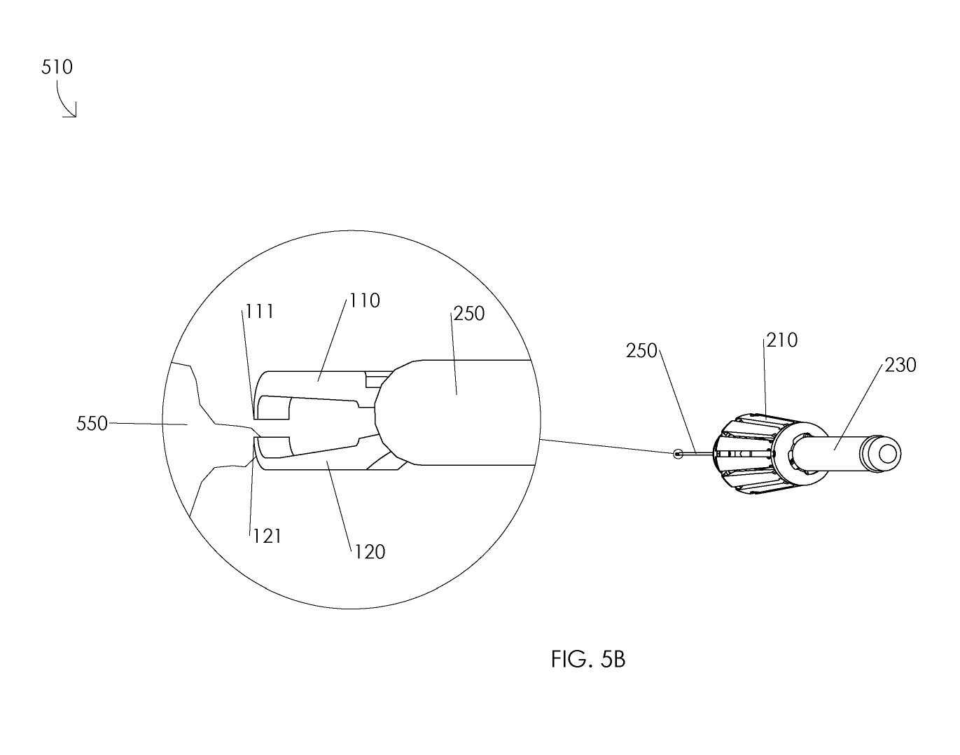

FIGS. 4A, 4B, and 4C are schematic diagrams illustrating a gradual closing of an asymmetric membrane removing forceps tip 100. FIG. 4A illustrates a top view of an open asymmetric membrane removing forceps 400. Illustratively, asymmetric membrane removing forceps tip 100 may comprise an open asymmetric membrane removing forceps 400 when actuation structure 210 is fully decompressed. In one or more embodiments, asymmetric membrane removing forceps tip 100 may comprise an open asymmetric membrane removing forceps 400 when stationary medial face 115 is fully separated from actuating medial face 125, e.g., asymmetric membrane removing forceps tip 100 may comprise an open asymmetric membrane removing forceps 400 when stationary medial face 115 is separated from actuating medial face 125 by medial face separation distance 136. Illustratively, a line normal to a surface of actuating medial face 125 may intersect a line normal to a surface of stationary medial face 115 at an angle when asymmetric membrane removing forceps tip 100 comprises an open asymmetric membrane removing forceps 400.

FIG. 4B illustrates a top view of a partially closed asymmetric membrane removing forceps 410. Illustratively, a compression of actuation structure 210 may be configured to gradually close asymmetric membrane removing forceps tip 100 from an open asymmetric membrane removing forceps 400 to a partially closed asymmetric membrane removing forceps 410, e.g., asymmetric membrane removing forceps tip 100 may comprise a partially closed asymmetric removing forceps 410 when actuation structure 210 is partially compressed. In one or more embodiments, a compression of actuation structure 210 may be configured to extend hypodermic tube 250 over a portion of stationary forceps jaw 110 and over a portion of actuating forceps jaw 120. Illustratively, an extension of hypodermic tube 250 over a portion of stationary forceps jaw 110 and over a portion of actuating forceps jaw 120 may be configured to actuate actuating forceps jaw 120 relative to stationary forceps jaw 110. In one or more embodiments, a compression of actuation structure 210 may be configured to actuate actuating forceps jaw 120 relative to stationary forceps jaw 110 wherein a separation distance between stationary medial face 115 and actuating medial face 125 is gradually decreased. Illustratively, a compression of actuation structure 210 may be configured to gradually close asymmetric membrane removing forceps tip 100 wherein only actuating forceps jaw 120 actuates in a transverse plane of actuation structure 210, e.g., a compression of actuation structure 210 may be configured to gradually close asymmetric membrane removing forceps tip 100 wherein stationary forceps jaw 110 does not actuate in a transverse plane of actuation structure 210. In one or more embodiments, a line normal to a surface of actuating medial face 125 may be parallel to a line normal to a surface of stationary medial face 115 when asymmetric membrane removing forceps tip 100 comprises a partially closed asymmetric removing forceps 410. Illustratively, a compression of actuation structure 210 may be configured to cause a line normal to a surface of actuating medial face 125 that intersects a line normal to a surface of stationary medial face 115 at an angle to gradually become parallel to the line normal to the surface of stationary medial face 115.

FIG. 4C illustrates an isometric view of a fully closed asymmetric membrane removing forceps 420. Illustratively, a compression of actuation structure 210 may be configured to gradually close asymmetric membrane removing forceps tip 100 from a partially closed asymmetric membrane removing forceps 410 to a fully closed asymmetric membrane removing forceps 420, e.g., asymmetric membrane removing forceps tip 100 may comprise a fully closed asymmetric removing forceps 420 when actuation structure 210 is fully compressed. In one or more embodiments, actuating medial face 125 may contact stationary medial face 115 when asymmetric membrane removing forceps tip 100 comprises a fully closed asymmetric removing forceps 420. Illustratively, actuating medial face 125 may contact stationary medial face 115 wherein actuating medial face distal end 126 is adjacent to stationary medial face distal end 116 when asymmetric membrane removing forceps tip 100 comprises a fully closed asymmetric removing forceps 420. In one or more embodiments, actuating medial face 125 may contact stationary medial face 115 wherein actuating medial face proximal end 127 is adjacent to stationary medial face proximal end 117 when asymmetric membrane removing forceps tip 100 comprises a fully closed asymmetric removing forceps 420. Illustratively, actuating medial face 125 may contact stationary medial face 115 wherein actuating medial face distal end 126 is adjacent to stationary medial face distal end 116 and wherein actuating medial face proximal end 127 is adjacent to stationary medial face proximal end 117 when asymmetric membrane removing forceps tip 100 comprises a fully closed asymmetric removing forceps 420. In one or more embodiments, a portion of stationary base 119 may contact a portion of actuating curved base 129 when asymmetric membrane removing forceps tip 100 comprises a fully closed asymmetric removing forceps 420, e.g., a portion of stationary base 119 may contact a portion of actuating curved base 129 and actuating medial face 125 may contact stationary medial face 115 forming a fully enclosed opening between actuating forceps jaw 120 and stationary forceps jaw 110 when asymmetric membrane removing forceps tip 100 comprises a fully closed asymmetric removing forceps 420. Illustratively, actuating curved base distal end 128 may be configured to extend a distance from hypodermic tube distal end 251 when asymmetric membrane removing forceps tip 100 comprises a fully closed asymmetric removing forceps 420. In one or more embodiments, lateral projection 118 may be configured to extend a distance from hypodermic tube distal end 251 when asymmetric membrane removing forceps tip 100 comprises a fully closed asymmetric removing forceps 420. Illustratively, stationary forceps jaw proximal shoulder 145 and actuating forceps jaw proximal shoulder 155 may be adjacent when asymmetric membrane removing forceps tip 100 comprises a fully closed asymmetric removing forceps 420.

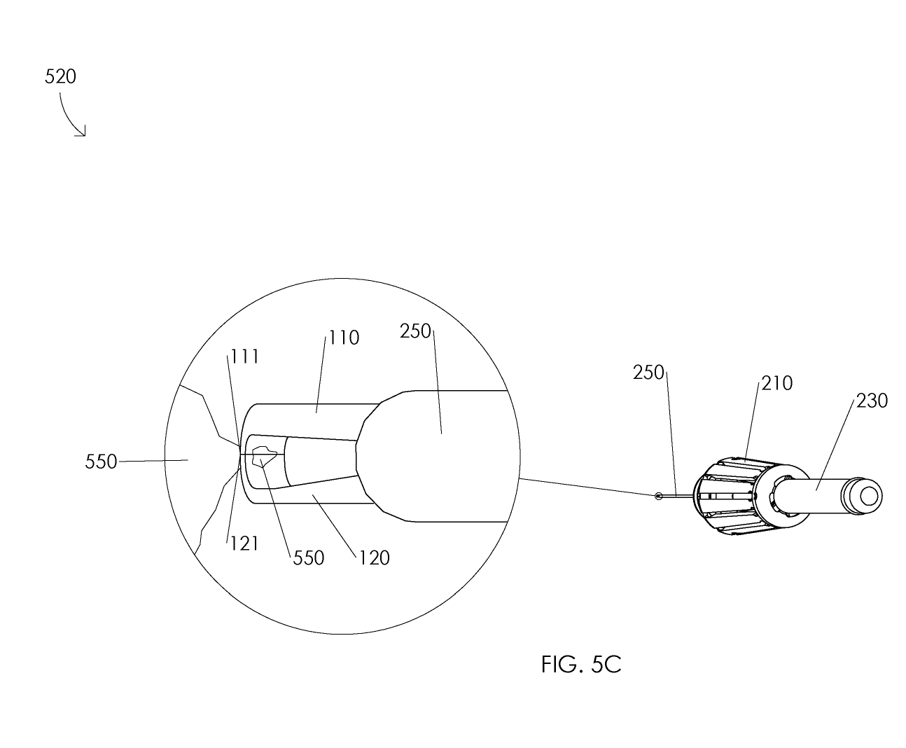

FIGS. 5A, 5B, and 5C are schematic diagrams illustrating a surgical procedure. FIG. 5A illustrates a membrane approach 500. Illustratively, a membrane approach 500 may be achieved through a cannula disposed in an incision in a pars plana of an eye. In one or more embodiments, a surgeon may begin a membrane approach 500 by inserting asymmetric membrane removing forceps tip 100 and hypodermic tube 250 into a cannula and advancing asymmetric membrane removing forceps tip 100 into an inner portion of an eye until stationary forceps jaw distal end 111 and actuating forceps jaw distal end 121 approach a retina. Illustratively, membrane 550 may be disposed over a portion of a retina. In one or more embodiments, membrane 550 may comprise an internal limiting membrane. Illustratively, membrane 550 may comprise an epiretinal membrane.

FIG. 5B illustrates a partial membrane grasping 510. Illustratively, a surgeon may perform a partial membrane grasping 510 by aligning stationary forceps jaw distal end 111 over an edge of membrane 550 and aligning actuating forceps jaw distal end 121 over a portion of membrane 550. In one or more embodiments, a surgeon may compress actuation structure 210 to gradually actuate actuating forceps jaw distal end 121 over membrane 550. Illustratively, an actuation of actuating forceps jaw distal end 121 over membrane 550 may be configured to raise a portion of membrane 550. In one or more embodiments, a raised portion of membrane 550 may be swept by actuating medial face 125 towards stationary medial face 115 as a distance between actuating medial face 125 and stationary medial face 115 decreases. Illustratively, an amount of membrane 550 disposed between actuating medial face 125 and stationary medial face 115 may increase as a distance between actuating medial face 125 and stationary medial face 115 decreases.

FIG. 5C illustrates a complete membrane grasping 520. Illustratively, a compression of actuation structure 210 may be configured to gradually close asymmetric membrane removing tip 100 from a partial membrane grasping 510 to a complete membrane grasping 520. In one or more embodiments, a surgeon may perform a complete membrane grasping 520 without actuating stationary forceps jaw 110 relative to membrane 550. Illustratively a surgeon may perform a complete membrane grasping 520 by only actuating actuating forceps jaw 120 relative to membrane 550. In one or more embodiments, performing a complete membrane grasping 520 by actuating only actuating forceps jaw 120 relative to membrane 550 and not actuating stationary forceps jaw 110 relative to membrane 550 may be configured to reduce a risk of trauma to an underlying retina. Illustratively, attempting to perform a complete membrane grasping 520 by actuating only actuating forceps jaw 120 relative to membrane 550 and not actuating stationary forceps jaw 110 relative to membrane 550 may be configured to reduce a difficulty of performing a complete membrane grasping 520.

The foregoing description has been directed to particular embodiments of this invention. It will be apparent; however, that other variations and modifications may be made to the described embodiments, with the attainment of some or all of their advantages. Specifically, it should be noted that the principles of the present invention may be implemented in any system. Furthermore, while this description has been written in terms of a membrane removing forceps, the teachings of the present invention are equally suitable to any systems where the functionality may be employed. Therefore, it is the object of the appended claims to cover all such variations and modifications as come within the true spirit and scope of the invention.

What is claimed is:

1. An instrument comprising:
an actuation structure having an actuation structure distal end and an actuation structure proximal end;
a hypodermic tube having a hypodermic tube distal end and a hypodermic tube proximal end, the hypodermic tube proximal end disposed in a hypodermic tube housing of the actuation structure;
a blank having a blank distal end and a blank proximal end, the blank disposed within the actuation structure and the housing tube;
a stationary forceps jaw of the blank having a stationary forceps jaw distal end and a stationary forceps jaw proximal end;
an actuating forceps jaw of the blank having an actuating forceps jaw distal end and an actuating forceps jaw proximal end;
an asymmetric aperture in the blank, the asymmetric aperture disposed laterally relative to a midline of the blank;
is a stationary base of the stationary forceps jaw; and
an actuating curved base of the actuating forceps jaw having an actuating curved base distal end and an actuating curved base proximal end.

2. The instrument of claim 1 wherein a compression of the actuation structure is configured to actuate the actuating forceps jaw relative to the stationary forceps jaw.

3. The instrument of claim 2 wherein the compression of the actuation structure is configured not to actuate the stationary forceps jaw.

4. The instrument of claim 1 further comprising:
a lateral projection of the stationary forceps jaw, the lateral projection extending from the stationary base at an angle normal to a surface of the stationary base.

5. The instrument of claim 1 further comprising:
a stationary medial face of the stationary forceps jaw having a stationary medial face distal end and a stationary medial face proximal end; and
an actuating medial face of the actuating forceps jaw having an actuating medial face distal end and an actuating medial face proximal end.

6. The instrument of claim 5 wherein a compression of the actuation structure is configured to actuate the actuating medial face towards the stationary medial face.

7. The instrument of claim 6 wherein the compression of the actuation structure is configured not to actuate the stationary medial face.

8. The instrument of claim 1 further comprising:
a stationary forceps jaw proximal shoulder of the stationary forceps jaw; and
a stationary forceps jaw distal shoulder of the stationary forceps jaw.

9. The instrument of claim 1 further comprising:
an actuating forceps jaw proximal shoulder of the actuating forceps jaw; and
an actuating forceps jaw distal shoulder of the actuating forceps jaw.

10. The instrument of claim 9 further comprising:
an actuating forceps jaw medial discontinuity of the actuating forceps jaw, the actuating forceps jaw medial discontinuity disposed between the actuating forceps jaw proximal shoulder and the actuating forceps jaw distal shoulder.

11. The instrument of claim 1 further comprising:
a removable handle having a removable handle distal end and a removable handle proximal end, a portion of the removable handle disposed in the actuation structure.

12. The instrument of claim 11 wherein the portion of the removable handle is temporarily fixed in the actuation structure by a torsional snap fit.

13. An instrument comprising:
an actuation structure having an actuation structure distal end and an actuation structure proximal end;
a hypodermic tube having a hypodermic tube distal end and a hypodermic tube proximal end, the hypodermic tube proximal end disposed in a hypodermic tube housing of the actuation structure;
a blank having a blank distal end and a blank proximal end, the blank disposed within the actuation structure and the housing tube;
a stationary forceps jaw of the blank having a stationary forceps jaw distal end and a stationary forceps jaw proximal end;
a stationary medial face of the stationary forceps jaw having a stationary medial face distal end and a stationary medial face proximal end;
an actuating forceps jaw of the blank having an actuating forceps jaw distal end and an actuating forceps jaw proximal end;
is an actuating medial face of the actuating forceps jaw having an actuating medial face distal end and an actuating medial face proximal end;
an asymmetric aperture in the blank, the asymmetric aperture disposed laterally relative to a midline of the blank;
a stationary base of the stationary forceps jaw; and
an actuating curved base of the actuating forceps jaw having an actuating curved base distal end and an actuating curved base proximal end.

14. The instrument of claim 13 wherein a compression of the actuation structure is configured to actuate the actuating forceps jaw.

15. The instrument of claim 14 wherein the compression of the actuation structure is configured not to actuate the stationary forceps jaw.

16. The instrument of claim 15 wherein the compression of the actuation structure is configured to actuate the actuating medial face towards the stationary medial face.

17. The instrument of claim 13 further comprising:
a lateral projection of the stationary forceps jaw, the lateral projection extending from the stationary base at an angle normal to a surface of the stationary base.

18. The instrument of claim 13 further comprising:
a removable handle having a removable handle distal end and a removable handle proximal end, a portion of the removable handle disposed in the actuation structure.

19. The instrument of claim 18 wherein the portion of the removable handle is temporarily fixed in the actuation structure by a torsional snap fit.

20. An instrument comprising:
an actuation structure having an actuation structure distal end and an actuation structure proximal end;
a removable handle having a removable handle distal end and a removable handle proximal end, a portion of the removable handle disposed in the actuation structure;
a hypodermic tube having a hypodermic tube distal end and a hypodermic tube proximal end, the hypodermic tube proximal end disposed in a hypodermic tube housing of the actuation structure;
a blank having a blank distal end and a blank proximal end, the blank disposed within the actuation structure and the housing tube;
a stationary forceps jaw of the blank having a stationary forceps jaw distal end and a stationary forceps jaw proximal end;
a stationary medial face of the stationary forceps jaw having a stationary medial face distal end and a stationary medial face proximal end;
an actuating forceps jaw of the blank having an actuating forceps jaw distal end and an actuating forceps jaw proximal end;
an actuating medial face of the actuating forceps jaw having an actuating medial face distal end and an actuating medial face proximal end;
an asymmetric aperture in the blank, the asymmetric aperture disposed laterally relative to a midline of the blank;
a stationary base of the stationary forceps jaw; and
an actuating curved base of the actuating forceps jaw having an actuating curved base distal end and an actuating curved base proximal end.
